(19) United States
(12) Patent Application Publication
(10) Pub. No.: US 2002/0128575 A1
(43) Pub. Date: Sep. 12, 2002

(54) Stepney limb elevator, a limb support apparatus

(76) Inventors: Marie Antionette Robinson, Stone Mountain, CA (US); Isaiah Callahan, Atlanta, GA (US)

Correspondence Address: Marie Antionette Robinson, 5001 W. Mountain Street, Stone Mountain, CA, 30083, US

(21) Appl. No.: 09/905,778

(22) Filed: Jul. 14, 2001

Related U.S. Application Data
(60) Provisional application No. 60/218,514, filed on Jul. 15, 2000.

(51) Int. Cl. A61F 5/00

(52) U.S. Cl. 602/32

(57) ABSTRACT

A new and improved limb support apparatus that include a vertical support assembly, embodying a top portion/female part, which is adapted to serve as a cradle or rest for the limb. A bottom portion/male part that add stability to the vertical support assembly, possessing a wide base and a profound fit within the top portion. Both, the top segment and the bottom segment have correlating holes or openings in which a pin is selectively inserted creating the locking assembly.

### BACKGROUND OF THE INVENTION

1. Field of the Invention

The present invention relates generally to limb supports, more specifically a fulcrum that lifts and assists in maintaining in place an extremity (arm or leg) while medical treatment is applied.

2. Description of the Prior Art

In the past, medical personnel (nurses, physicians, therapists etc.) are incapable of elevating a limb effectively to administer dressings, when they are prescribed course of treatment. Arms and legs are manually held (usually placed under the arm of the individual administering the treatment) or propped on bed pillows, folded sheets or Styrofoam forms. If the limb is heavy, pillows, bed sheets nor Styrofoam are firm enough to support the affected part and have it elevated to the desired height. Manually holding in place is almost impossible. To avoid the use of bed pillows, bed sheets, Styrofoam forms or individual manipulation, additional personnel are utilized to hold the limb. Only then are both hands free to apply the ordered coverings, protective or supportive, to the diseased or injured part. Strict aseptic technique must be followed during dressing changes to avoid infection. When sponges or gauze are dropped, they are discarded and new materials must be used. During this procedure there is an increased risk for infection if new supplies are not utilized. The skill in performing this task is compromised when treatment using pillows, bed sheets, Styrofoam forms or individual manipulation is used. If drainage occurs during the procedure, soiling may occur, adding to the risk of infection. The pillows, their coverings and sheets must be laundered. The Styrofoam forms, eventually are discarded. Anti-disinfectives are usually prescribed. Some are staining when spilled; again laundering and discarding are the options.

Thus, while foregoing efforts indicate it to be well known that elevation through support (regarding dressing changes) produces quality patient care, the prior efforts described herein fall short of this realization. A support with the following desirable features is suggested: 1. permit the free movement of the hands allowing for the proper application of the prescribed treatment and eliminates the need for additional personnel to hold the limb when two (pair) are used; 2. made of plastic, it can be cleaned with soap and water or any type of anti-bacterial agent; 3. is light weight and 4. can be produced in various sizes.

The following desired characteristics are provided by the unique support of the present invention.

### SUMMARY OF THE INVENTION

To accomplish the foregoing, the present invention, briefly described, provides a new and improved limb support apparatus, which includes a vertical fulcrum assembly. It consists of a U-shaped top portion (female), adapted to serve as a cradle or rest for the limb. It is curved with increased breadth to prevent bruising or markings on the extremity. Also at the bottom of the top portion or female part, are holes, spaced apart for a locking assembly. The bottom portion or male part of the support apparatus extends well into the top portion for additional stability. It too, possesses holes/openings, corresponding exactly to the female portion or top part of the device. The bottom/male portion of the fulcrum includes a pin adapted to be selectively inserted into the correlating openings in the bottom/male, top/female to achieve the desired height and lock the apparatus in place. The base portion of the bottom/male part extends beyond the U-shaped top/female part increasing sturdiness.

This brief description gives a rather broad account of the important attributes of the present invention.

It is the purpose of the present invention to provide a new and improved limb support and none of the hindrances associated with the prior art.

It is the purpose of the present invention to provide an economically produced limb support, which is durable and dependable.

It is the purpose of the present invention to permit the use of hands, when dressings are the prescribed course of treatment.

It is another object of the present invention to be mass-produced in various sizes.

Still another object of the present invention is to provide a new and improved support for limbs that can adjust in height.

Another object of the present invention is its cleanability. It can be cleaned with soap and water or any type of antibacterial agent.

Still, a further object of the present invention is that the support is light weight and can be easily carried.

For a much better understanding of the invention and its specific advantages attained by use, reference should be made to the accompanying drawings and the manner in which they are depicted.

### BRIEF DESCRIPTION OF THE DRAWINGS

The invention will be better comprehended after a study of the following illustrations. The aforesaid objects and other objects will become more easily understood. The description regarding the attached drawing wherein:

FIG. 1 is a front perspective view showing the cradle/rest, the vertical support assembly and the base

FIG. 2 is a rear view perspective view also showing the cradle/rest, the vertical support assembly and the base.

FIG. 3 is an enlarged view of the entire fulcrum. The male/bottom portion fits deeply into the top/female part for greater stability.

FIG. 4 is an enlarged view of the U-shaped cradle/rest. Curved, it illustrates the breadth of the top/female portion shown in FIG.4.

FIG. 5 is an enlarged view of the locking assembly of the limb support apparatus of FIG.4. The pin is inserted in the locking position.

FIG. 6 is the base of the preferred embodiment of the limb support apparatus shown in FIG.4. It extends beyond the U-shaped-top/female portion.

### DESCRIPTION OF THE PREFERRED EMBODIMENT

Referring to the illustrations, a new and improved limb support apparatus representing the principles and ideas of the present invention will be described as follows:

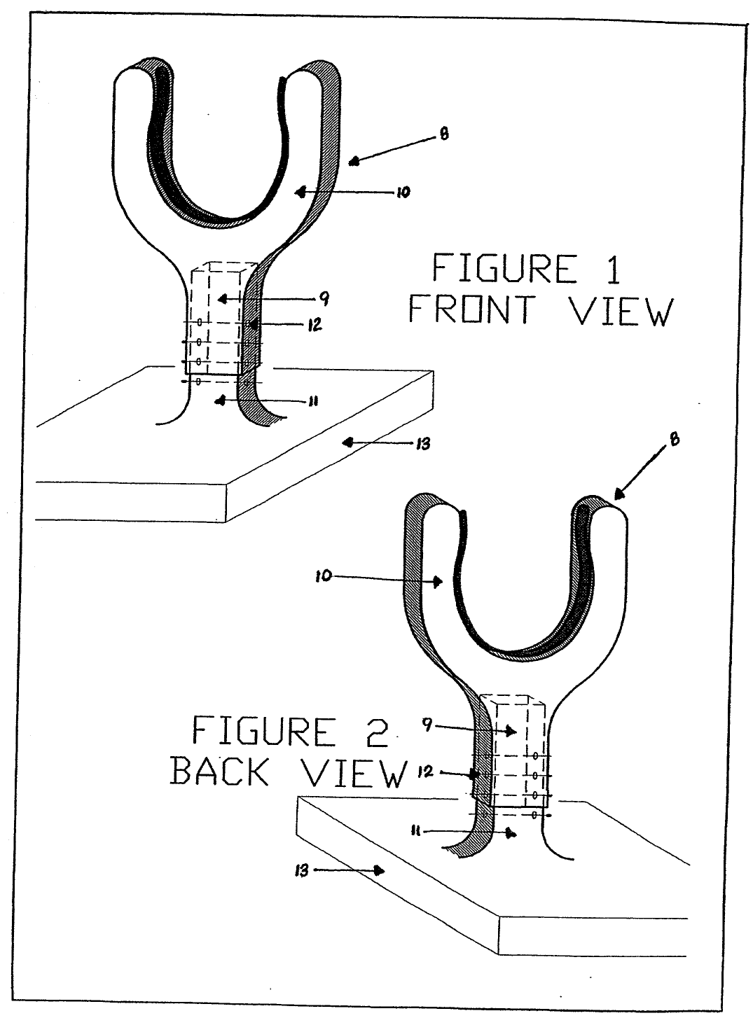

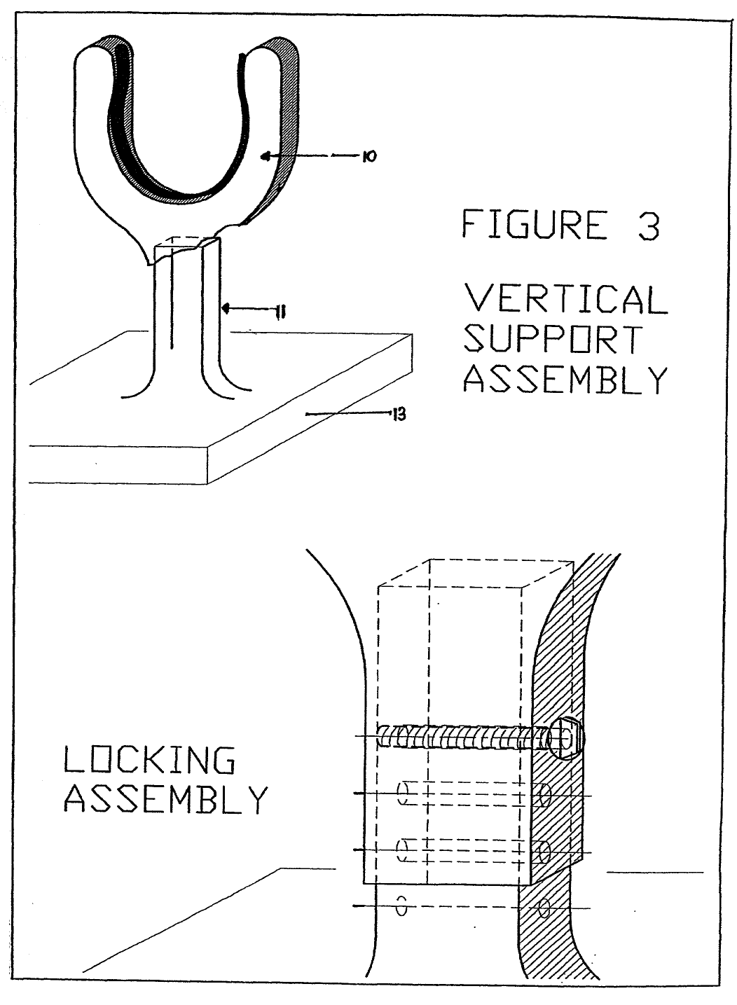

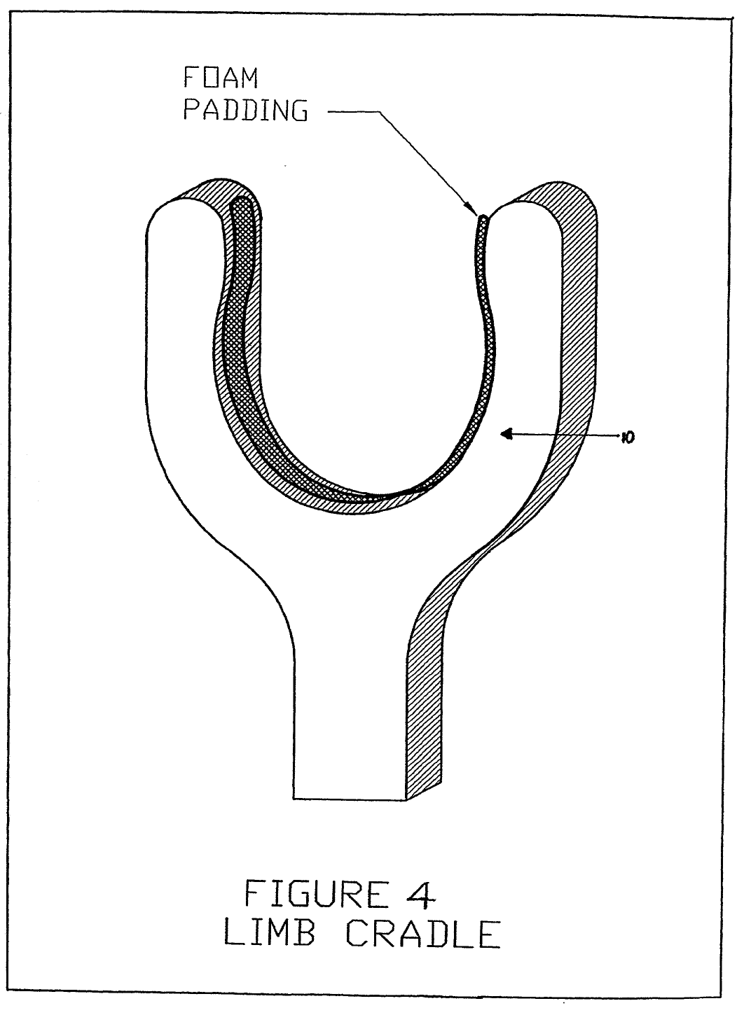

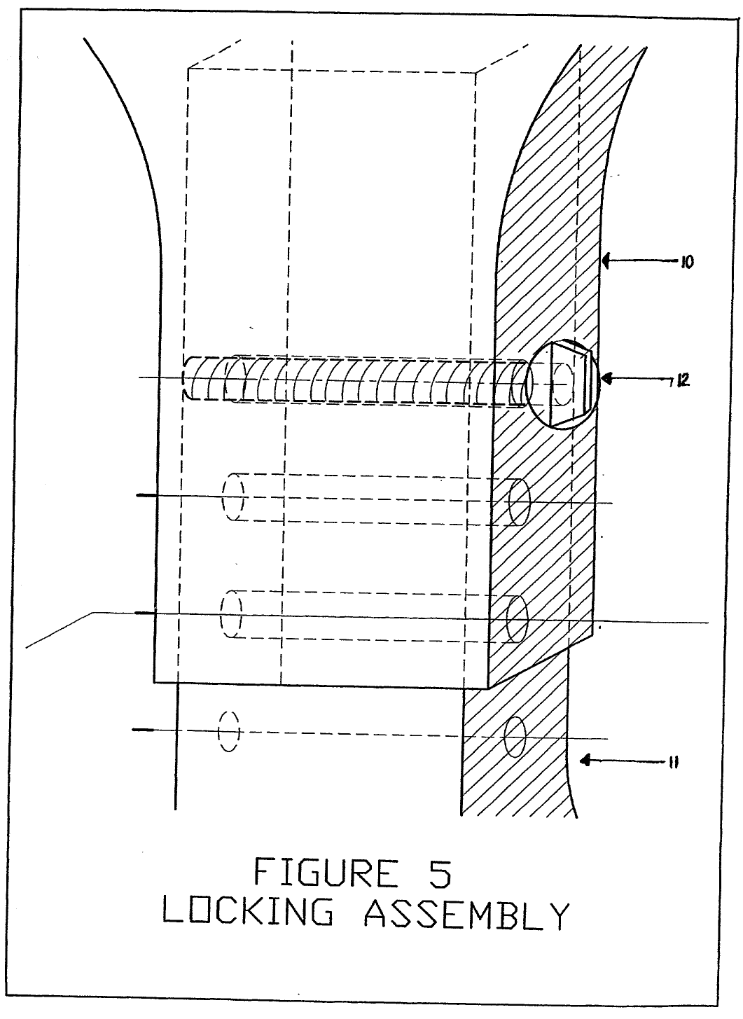

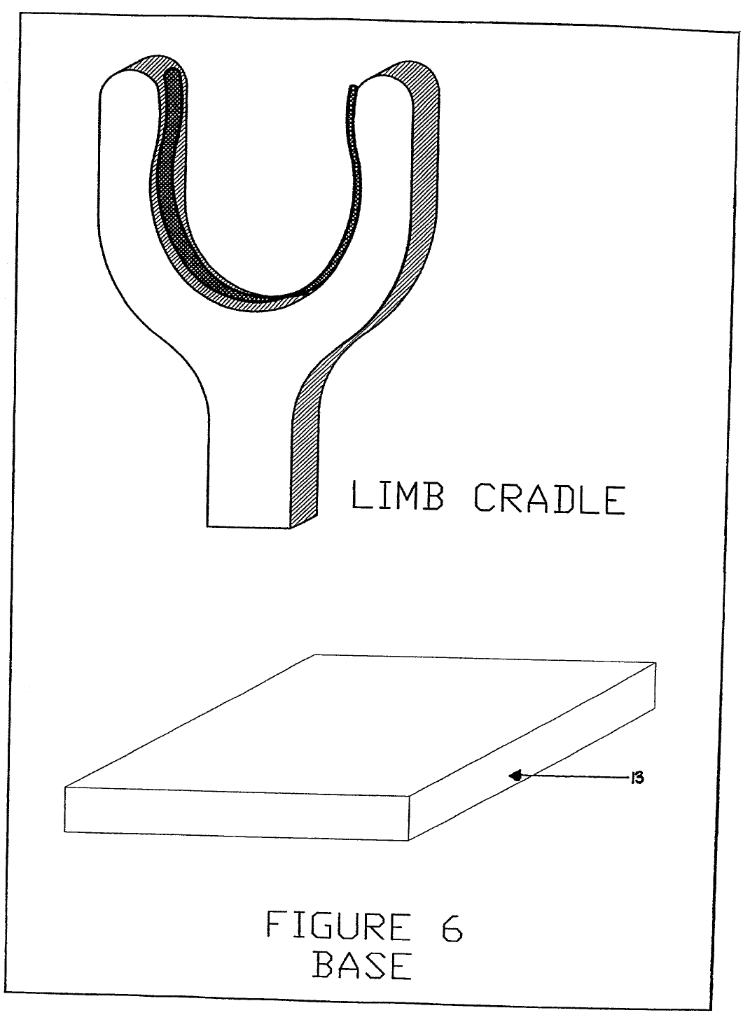

FIGS. 1-6, shows an extraordinary embodiment of the limb support apparatus of the invention, designated by reference numeral 8. In its preferred form, limb support apparatus 8 includes a vertical support assembly 9, which includes a top/female portion 10 adapted to serve as a cradle/rest, below and inside the top/female portion 10 is the bottom/male portion, including base 11. A locking assembly 12 includes the holes/openings in which the pin is selectively inserted. And 13 is the base in which the vertical fulcrum 9 rests.

The components of the limb support apparatus of the invention can be made from inexpensive plastic material.

The present invention accomplishes all objects set forth by giving a new and improved limb support at relatively low cost, simplistic in design and operation. With the use of this invention a limb is supported. The hands have free movement. With this invention clean up is easy and is lightweight. With this invention various sizes can be produced. With this invention the use of additional personnel is not needed. With its utilization, savings in supplies, time and personnel add up to economical savings that can be realized almost immediately.

What is claimed as being new and desired to be protectected Letters Patent of the united states is as follows:

1. A new and improved limb support apparatus,comprising:
a vertical support assembly which includes a top/female portion adapted to serve as a cradle/rest, with cushion material lining portion, below and inside the top/female portion is the male portion including the base.
a locking assembly includes the holes/openings in which the pin is selectively inserted. a base in which the vertical fulcrum rests.

2. The apparatus described in claim 1 wherein:
said top portion of said vertical support assembly included a U-shaped, cradle/rest, with cushion material and a top portion of the locking assembly, which includes holes/ openings in which a pin is, selectively inserted.

3. The apparatus described in claim 1 wherein:
said bottom portion of said vertical support assembly included the bottom portion of the of the locking assembly that fits inside the top/female portion. The bottom part also contain holes/openings that align with the holes/openings in the top portion, in which the pin is selectively inserted. The bottom/male portion includes the base in which the apparatus described in claim 2 rests.

4. The apparatus described in claim 1 wherein:
said locking assembly of said vertical support assembly includes holes/openings of the top/female portion and the holes/openings of the bottom/male portion. The holes in the top/female portion align with the bottom/male portion in which a pin is selectively inserted.
